(19) United States
(12) Patent Application Publication
(10) Pub. No.: US 2013/0110027 A1
(43) Pub. Date: May 2, 2013

(54) MULTI-PURPOSE MODULAR TRAUMA MANAGEMENT SYSTEM AND NOVEL INDIVIDUAL ELEMENTS FORMING PART THEREOF

(71) Applicant: Anthony Philip Kobler, Cherrybrook (AU)

(72) Inventor: Anthony Philip Kobler, Cherrybrook (AU)

(21) Appl. No.: 13/285,494

(22) Filed: Oct. 31, 2011

(51) Int. Cl. A61F 13/00

(52) U.S. Cl. 602/75

(57) ABSTRACT

A modular trauma management system is provided which includes a number of multi-purpose elasticised pressure and/or support bandages combined with a modular framing support system based on a series of rods or tubes and connectors. The bandages are connectable lengthwise to like bandages to form longer bandage lengths and the modular framing system can be used to create numerous forms of splints, supports structures, crutches, walking sticks and the like. The system may include additional multi use elements that can further interact with the bandage and framing system elements such as multi-function tourniquet straps, modular stretcher elements, a traction strapping system and a versatile sling system that includes a separate multi use strap element. These components interact with the other elements in the system to enable a lightweight, highly versatile, environmentally tolerant system that can be used almost anywhere to manage an extremely wide range of trauma situations.

### FIELD OF THE INVENTION

The present invention relates to a kit form multi-purpose trauma management system and to the novel and inventive individual elements of that system. The invention has been developed to provide a lightweight, compact, transportable full range trauma management system, particularly suited for use in remote locations without vehicular support, and will be described herein with reference to this preferred application. However, it will be appreciated by those skilled in the art, that the full kit and/or individual elements or various sub-combinations of elements, are equally suited to use in wider more general first aid applications such as when travelling, camping, at work, at sporting or adventure activities or in home environments.

### BACKGROUND OF THE INVENTION

Any discussion of the prior art throughout the specification should in no way be considered as an admission that such prior art is widely known or forms part of common general knowledge in the field.

When travelling in remote inaccessible areas, such as during armed forces exercises or manoeuvres, or trekking on an expedition in hazardous terrain, it is desirable to have adequate trauma management equipment to cover all possible trauma eventualities. In fact, in some jurisdictions, local legislation, such as that relating to Occupational Health and Safety (OH&S), requires minimum levels of equipment for commercial ventures. Meeting these needs or requirements using currently available equipment will generally necessitate putting together an extensive kit of equipment that includes the following items or a similar combination of items:
a range of bandages to treat general bleeding;
a long elasticated bandage, plus immobilisation device, to treat bites and stings;
tourniquet equipment for extreme fluid stop applications;
neoprene or elastic supports to treat sprains to joints;
various triangular bandages for upper arm injuries;
additional bandages and flexible arm splint for lower arm injuries;
a vacuum splint for shoulder dislocation;
an inflatable splint or similar for lower leg injury;
a femur traction device for upper leg injury;
multiple gauze bandages for chest injury support; and
a stretcher for conveying the victim if they have been immobilized.

Many of these components, particularly the splint kits and stretcher, are each designed for a single purpose or use and are individually quite cumbersome. In many instances the overall weight and volume of a full set of equipment such as this means that it isn't always feasible to take the full complement, and compromises have to be made.

Often, a kit has to be rationalised to a conventional set of basic bandages and gauzes and rescuers have to rely on trying to locate splints and/or stretcher supports in the field, which is far from ideal and for many situations clearly inadequate.

In this regard, general trauma management and teaching hasn't changed very much over the last few decades. While there have been significant technological advancements in relation to individual trauma-specific items of equipment designed for specific interventions, it appears that until now, little advance has been made in terms of the development of multi use interacting components that can work together to rationalise the components required for a wide range of trauma events.

It is an object of the present invention to provide a multi-purpose modular trauma management system and/or novel individual elements that overcome or ameliorate one or more of the above-discussed disadvantages of the prior art, or which at least offer a useful alternative.

### SUMMARY OF THE INVENTION

Pressure and/or Support Bandage

According to a first aspect of the invention there is provided a pressure and/or support bandage comprising:

an elongate strip of elastically extendible material having a length L extending from a first end to a second end and a width W, with a first surface on one side and a second surface on the remote side;

at least one region of hook fastener material or equivalent extending substantially across the width W at least partially on the first surface at or adjacent one of said first or second ends; and

a complementary at least one region of loop fastener material or a connectable material adapted to engage with said hook fastener material extending substantially across the width W at or adjacent the other of said first or second ends on the second surface on the remote side; the majority of the remainder of said second surface generally being of a connectable material adapted to engage with said hook fastener material;

whereby in use the bandage can be wound around an article and secured by connection of one or more of the hook fastener regions or equivalent on the first surface, with one or more of the complementary loop fastener regions on the remote surface and/or said connectable material, or alternatively, extended in length by overlapping end to end connection of a like bandage via engagement of at least one hook fastener region on one bandage with the corresponding complementary loop fastener or connectable material region on the remote surface of the other bandage.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

Preferably, the hook and/or loop fastener regions are in the form of strips of hook or loop fastener material respectively.

Desirably, the bandage includes at least two spaced strips of hook fastener material on the first surface at one end, and a corresponding at least two similarly spaced strips of loop material on the second surface at the other end.

In one preferred form the bandage further includes two or more pockets adapted to receive and retain splinting elements or other supports. Preferably, the pockets are provided as spaced apart pairs with opposed openings adapted to receive respective ends of a support rod that extends generally along length L.

Ideally, the width of the bandage is selected to be wide enough for pressure and/or support applications to large areas including limbs, stomach and chest. In preferred forms the width W is between 100 mm and 250 mm and more ideally around 150 mm.

Similarly, the length L is preferably selected to suit a combination of limb circumferences, and/or sized for other intended uses such as in splinting applications. Desirably the lengths range from around 400 mm to 900 mm, with currently preferred lengths of 500 mm or 600 mm. Optionally, a kit may comprise a selection of these bandages of differing lengths. In one particularly preferred current form, a kit includes bandage lengths of 500 mm and 600 mm.

The elongate strip of elasticised material can comprise any of a range of suitable materials that provide the requisite combination of resilience, durability, physical support, connectability with loop fastener material on at least one side, and ease of use. in the preferred form the material is a knitted waistband or plain knitted support elastic which by its structure is inherently securable across its surfaces with hook fastener material. The currently preferred form is a C930 150 mm wide strong plain knitted support elastic as described by the manufacturer Webbing industries Australia P/L.

Similarly, while in the preferred form a single material is used for the bandage which has uniform characteristics across its length and width, the strip could be of a composite construction fabricated from a combination of different materials, or be made of a material that has different properties at different locations and/or on different surfaces. For example, the material may only be hook fastener engageable on one surface.

The bandage system of the present invention therefore has the advantages of being easily applied and securable, and can be used individually or in combination with other like bandages to treat a wide range of injuries including those which require pressure and/or support and/or interaction with splint components and the like.

Modular Medical Support Framing System

According to a second aspect of the invention there is provided a modular medical support framing system adapted for creating a multiple range of splints, support frames and crutch type devices, the framing system comprising;

a plurality of different lengths of rods or tubing adapted at each end shaped to receive an inline connector piece or corner connector; and

a plurality of inline and corner connector connectors;

wherein the inline and corner connectors and/or rod ends are configured to prevent relative rotation of interconnected rods or tubing after connection.

Desirably, the system further includes a plurality of adjustable angle connectors.

In one preferred form the rods comprise multiple different length square section tube elements, preferably made from aluminium or a lightweight high strength alloy. In the currently preferred form the tubes are made from aluminium, preferably of square section, and have an external size of 16 mm and a wall thickness of around 1.5 mm. Clearly other shapes and sizes may be suitable.

The preferred inline and corner connectors are made from moulded glass-reinforced nylon (other materials could clearly be suitable) and include a centralised corner element or connector stop formation from which extend two male lugs designed to provide an interference fit with the square hollow ends of the preferred tube elements described above. In such instances the lugs are preferably tapered at their distal ends to enable easy initial connection.

However, other connector structures could be suitable including connectors configured to connect with the outside surfaces of the rod elements.

The resistance to relative rotation between interconnected rod elements and the connectors themselves can be achieved purely by selection of the elements inherent shape, as is the case when using square section hollow tubing, or can be achieved via incorporation of other non-circular connecting faces or locking pins etc.

Ideally, the spacing of the pairs of pockets, optionally provided on the bandage of the first aspect of the invention, is configured to receive and retain specific unitary or conjoined rod elements from the modular framing system kit of components.

In one preferred embodiment the framing system kit includes the following basic components:

4×rods A−@ 250-300 mm (preferably 280 mm);

3×rods B−@ 120-150 mm (preferably 137.5 mm such that two plus joiner Is 280 mm);

2×rods C−@ 50-75 mm (preferably 66.25 mm such that two plus joiner is 137.5mm);

4×inline connectors D; and

4×corner connectors E.

Ideally the sizes are selected such that two B rods plus inline connector D equals the length of one A rod and 2 C rods plus 1 B rod plus two inline connectors D also equals one A rod etc.

This basic set is sufficient, in combination with the bandages of the first aspect of the invention and/or other straps including tourniquet straps and traction strap as hereinafter described, to produce numerous support structures a selection of which are outlined below:

Immobilising Splints/Support Frames:
support mat—bandage with pockets and 2 A rods & bandage to hold in place
immobilising splint—1 rod A or connected length of rods and 2 tourniquets with pockets and bandage to hold in place
immobilising splint—multiple rods A and B, 2 tourniquets with pockets, inline connector D, bandages to support to splint
pressure immobilisation system—joined bandages to create pressure bandage for full limb, multiple rods A, 2 tourniquets with pockets, inline connector D and third tourniquet to hold middle joint in place
immobilising splint frame—rods A and B, 2 tourniquets with pockets, inline and corner connectors D and E and bandage to hold in place
immobilising splint for shoulder—multiple rods A and B, inline and corner connectors D and E, tourniquets (at least one with pocket), bandages joined to hold in place around chest and bandages to support arm against splint
simple support platform—2 rods A, 2 rods B, 4 corner connectors E, bandage wrapped over frame and bandage to support limb to frame
support platform (many shapes available)—rods A and B, inline and corner connectors D and E, bandages wrapped around frame and bandages to support limb to frame
lateral femur traction splint—Tourniquet with pocket, rods A, B and C, inline and corner connectors D and E, foot strap, bandages to support leg to splint
emergency pole—rods A, inline connectors D

In a preferred form, the kit further comprises:

1×Padded rod @ approx. 150 mm F

Inclusion of this component enables at least the following additional structures:
walking stick—padded rod F, rods A and B, inline and corner connectors D and E, rubber boot
emergency crutch—padded rod F, rods A, B and C, inline and corner connectors D and E, rubber boot
medial femur traction splint—padded rod F, rods A, B and C, inline and corner connectors D and E, foot strap, bandages to support leg to splint

In yet other forms the kit also includes one or more adjustable angle connectors to further increase the versatility when it is desirable to erect complex angled support structures.

As will be seen, this modular medical support framing system is extremely compact in that it can be dismantled into a kit of relatively short lengths plus connectors, yet with ease can be transformed to provide whatever specific tailored structural support is required. This can be as a support splint or part of a support platform, immobilisation splint for various body parts including complex applications such as forming a splint where the arm is locked in a position extended above the head, or external support devices such as a walking stick with handle or a crutch with articulated handle and underarm support.

Modular Stretcher System

According to a third aspect of the invention there is provided a lightweight modular dismantlable length variable stretcher system comprising;

a plurality of rod or tubing elements adapted at each end to receive an inline connector piece or corner connector;

a plurality of inline and corner connectors; wherein the inline and corner connectors and/or rod ends are configured to prevent relative rotation of interconnected rods or tubing after connection;

the rod or tubing elements being interconnectable via the connectors to form a generally rectangular stretcher frame; and

two or more stretcher sling elements each having a width corresponding to the width of said rectangular frame and a length less than the maximum length of the longest intended length of the rectangular frame and including peripherally located longitudinally extending pockets through which the longitudinal rectangular frame elements can extend;

whereby two or more lengths of stretcher can be created by utilising more or less rod elements along the frame length and selecting the appropriate number of sling elements to suit the created frame length.

In one preferred form the rods comprise multiple different length square section tube elements, preferable made from aluminium or a lightweight high strength alloy. In the currently preferred form the tubes are made from aluminium and have an external section size of 16 mm and a wall thickness of around 1.5 mm. Again, other shapes and sizes could clearly be used.

The preferred inline and corner connectors are made from moulded glass-reinforced nylon and include a centralised corner or connector stop formation from which extend two male lugs designed to provide an interference fit with the square hollow ends of the tube elements. In such instances the lugs are preferably tapered at their distal ends to enable easy initial connection. However, other connectors could also be suitable including types which connect with the outside surfaces of the rod elements. The resistance to relative rotation between interconnected rod elements and the connectors themselves, particularly at corner connections, can be achieved purely by selection of the element's inherent shape, as is the case when using square section hollow tubing, or can be achieved via other non-circular connecting faces or locking pins etc.

In one preferred embodiment the stretcher kit includes the following components:

4×stretcher sling elements;

14×rods A @ 250-300 mm;

2×rods B @ 120-150 mm;

12×inline connectors; and

4×corner connectors.

It will be appreciated that by utilising say three or four stretcher sling strap elements of shorter length corresponding to single or multiples of the lengths of the longest rods, the stretcher sling length can be constructed to suit how many lengths of the longest rods are needed to support the victim to be conveyed.

In a preferred embodiment, the stretcher kit is provided as an add-on extension or adjunct to the modular medical support framing system of the second aspect of the invention described above. In this form, the stretcher kit is designed to utilise rod and connector elements that are compatible with, or the same as, some of the rod and connector elements of the framing system. In this case a lesser number of rod elements and connectors may be required in the add-on kit, on the basis that elements of the framing system can, optionally, be deployed as part of the stretcher system.

Modular Multi-Function Sling Assembly

According to a fourth aspect of the invention there is provided a modular multi-function sling assembly, the assembly comprising:

a support pocket having a first end to be located in use at or adjacent a wrist and a second end to be located in use at or adjacent an elbow;

the pocket including wrist end connection means and elbow end connection means; and

a separate strap element, the strap being adapted at each end for releasable connection with each of the wrist end and elbow end connection means.

Preferably, the support pocket is made from an element of ideally lightweight readily rollable support fabric foldable around a longitudinal axis to form the pocket.

Desirably, the wrist end and elbow end connection means are fixed to and form part of the pocket.

The strap element preferably comprises a simple length of webbing or similar material which is adapted at its ends for releasable connection with the wrist end and elbow end connection means of the support pocket.

In one preferred form, the wrist connection means on the sling comprises a pair of loops or rings connected to the edges of the support pocket, such that in use the pair of rings are aligned. Similarly, in the preferred form the elbow connection means includes a strip of webbing material or similar that connects the pocket edges to form a large single connection loop that can be secured with the sling strap at any convenient location. Preferably, the pairs of rings are also secured to the pocket using a band of webbing material attached to the support fabric which acts to reinforce the pocket as well as provide a means to secure the rings.

Desirably, the strap element is further constructed so as to be independently fastenable at each end to itself, so that it can also operate independently to provide additional functions such as serving as a “collar and cuff” sling.

In one particularly preferred form the strap is preferably made of a webbing material and incorporates on one surface relatively short lengths of one of a hook or loop fastening material, with discrete or continuous lengths of the other of said hook or loop fastening material disposed in the gap between. In this manner, the ends of the strap can be looped around a wrist and secured back to the body of the strap on which the complimentary fastener material is provided to form a “collar and cuff” sling.

Alternatively, one end of the strap can be looped through the respective paired connection loops on the sling pocket at the wrist end and looped around the webbing loop at the elbow end with the lose end of the strap at both ends being reconnected to itself, to thereby form either a support or elevation s ling, both of which are fully and easily adjustable via the paired loops fastener arrangement on the pocket. The strap can be connected over the extended webbing loop at the elbow with the strap positioned to the front or the rear so as to either pass easily over the upper arm, shoulder and the neck if the arm needs to be secured to the chest, or behind the arm and shoulder and around the neck if pressure on the arm, shoulder and/or neck is undesirable.

Multi-Function Strap that can Operate as a Tourniquet

According to a fifth aspect of the invention there is provided a multi-function strapping device that can act as a tourniquet, the strap comprising:

a length of substantially inextensible material having a first end and a second end remote the first end;

a female clip socket secured to a first end;

a slidably repositionable male clip element provided at a location between the first and second end; and

a rod receiving loop formation between said female and male socket and clip;

whereby the length of substantially inextensible material is selected, such that in use as a tourniquet after connection and tightening of the strap via operation of the clip elements, a suitable rod is inserted in the rod receiving loop and rotated to further tension the strap, the excess strap length can be used to secure the rod to retain the tension.

Desirably, the rod receiving loop formation is located toward the female clip socket.

Preferably, the substantially inextensible material is a webbing material. Desirably, the female socket is secured to the webbing by passing an end of the webbing through a socket connector loop and connecting that end a distance from the socket so that a webbing loop is provided and the rod receiving loop is secured to the outer portion of this webbing loop. In this manner, during tightening of the tourniquet using the rod in the rod receiving loop, the webbing in the loop can move through the female clip loop and so reduce the risks of pinching.

In a preferred form, the strap further includes one or more pocket formations such that the straps can also be used to stabilise various splinting frames and structures constructed using the framing elements of the framing system or stretcher system according to the second and third aspects of the invention respectively. Such pocket formations could be integral with or otherwise secured to the strap or be provided or formed as a separate component that could, for example, be slid onto the strap and so be repositionable along the strap length as required.

Advantageously, the multi-function strap can be easily connected to one or more additional multi-function straps via interconnection of respective male clip elements on one strap with a female clip socket on another strap, so as to instantly create significantly longer straps as required.

Traction Strapping System

According to a sixth aspect of the invention there is provided a traction strapping system comprising:

a releasably securable limb cuff arrangement for securing around a distal limb joint; and

at least one tensionable length adjustable traction strap assembly extendable away from said distal limb joint and terminating in a first connector formation for securement to a fixed frame element,

whereby the traction strap assembly is length adjustable by means of the strap passing through a sliding lock such that the free end of the strap then extends back towards the cuff element and terminates in a loop formation by which traction can be directly or indirectly applied.

In this manner, tension is created by pulling the strap towards the patient thus enabling self-application which can be extremely useful when needed.

Desirably, the releasably securable limb cuff is connected to the tensionable length adjustable traction strap assembly by means of a plurality of connector straps so that the foot can be held stable and pressure can be distributed across the cuff as required. In the preferred form the connector straps form a loop or series of loops, each secured at both ends to the cuff and connected to the traction strap, ideally through a fixed loop provided at the adjacent proximal end of that strap.

Preferably, the connector formation is a simple loop which can secure to the frame element by the frame element passing there through.

Modular Trauma Management System

According to a seventh aspect of the invention there is provided a modular trauma management system including:

a plurality of pressure and/or support bandages in accordance with a first aspect of the invention; and

a modular medical framing support system in accordance with a second aspect of the invention.

Preferably the system further includes one or more of the following additional elements:

a modular stretcher system in accordance with the third aspect of the invention;

a modular multi-function sling assembly in accordance with the fourth aspect of the invention;

a multi-function strap that can operate as a tourniquet in accordance with the fifth aspect of the invention; and

a traction strapping system in accordance with the sixth aspect of the invention.

It will be appreciated that not only does the modular trauma management system of this aspect of the invention enable a compact, lightweight and highly adaptable kit that is easily transported, the nature of each of the preferred elements is such that the entire system is unaffected by environmental conditions such as water, altitude and heat and so, unlike many existing systems, can be used almost anywhere.

### BRIEF DESCRIPTION OF THE DRAWINGS

Preferred embodiments of the invention will now be described, by way of example only, with reference to the accompanying representations in which:

FIG. 1 is a plan view of a first embodiment pressure and/or support bandage in accordance with a first aspect of the invention;

FIG. 2 is an inverted plan view of the pressure and/or support bandage shown in FIG. 1;

FIG. 3 is a side view of the pressure and/or support bandage shown in FIG. 1;

FIG. 4 is a perspective view of two like pressure and/or support bandages of a kind shown in FIG. 1 joined to form an extended bandage;

FIG. 5 is a plan view of an alternate embodiment of a pressure and/or support bandage in accordance with the first aspect of the invention incorporating pockets for receiving and retaining support frame rods, such as those in accordance with the second aspect of the invention;

FIG. 6 is a perspective view of a length of hollow rod forming part of a first embodiment modular medical support framing system in accordance with a second aspect of the invention;

FIG. 7 is a perspective view of a first embodiment inline connector piece for use with the hollow rod framing system element shown in FIG. 6;

FIG. 8 is a perspective view of a first embodiment corner connector piece or elbow for use with the hollow rod framing system element shown in FIG. 6;

FIG. 9 is a side view of a first embodiment adjustable angle connector piece forming part of a modular medical support framing system according to the second aspect of the invention suitable for use with the hollow rod framing system element shown in FIG. 6;

FIG. 10 is a side view of a padded rod forming an optional part of a modular medical support framing system according to the second aspect of the invention;

FIG. 11 is an end view of the padded rod shown in FIG. 10;

FIG. 12 is a plan view of a modular dismountable length variable stretcher according to a third aspect of the invention;

FIG. 13 is a top perspective view of the stretcher sling element forming part of the sling system shown in FIG. 12;

FIG. 14 is a top perspective view of a support sling pocket forming part of a modular multi-function sling assembly according to a fourth aspect of the invention, shown in the folded in use position;

FIG. 15 is a schematic opened plan view of the inside of the support sling pocket shown in FIG. 14;

FIG. 16 is a schematic opened plan view of the outside of the support sling pocket shown in FIG. 14;

FIG. 17 is a schematic plan view of a strap element for use singly or with the support sling pocket of FIGS. 14 to 16 to form a modular multi-function sling assembly according to a fourth aspect of the invention;

FIG. 18 is a schematic side view of the strap shown in FIG. 17;

FIG. 19 is a top perspective view of a multi-function strapping device that can act as a tourniquet in accordance with a fifth aspect of the invention;

FIG. 20 is a side perspective view of the multi-function strapping device shown in FIG. 19;

FIG. 21 is a schematic plan view of a traction strapping system according to a sixth aspect of the invention;

FIG. 22 is a plan view of a preferred embodiment modular trauma management system in accordance with a seventh aspect of the invention in a carry roll system illustrated in the open configuration;

FIG. 23 illustrates the use of the multi-purpose bandage of FIGS. 1 to 5 in use to manage a bleeding upper arm situation;

FIG. 24 illustrates use of the multi-function strap of FIGS. 18 and 19 applied as a tourniquet on the upper arm;

FIG. 25 illustrates the use of multiple bandages of FIGS. 1 to 5 joined to provide support for a cracked rib or chest injury situation;

FIG. 26 illustrates the same joined bandages in use for treatment of abdominal problems;

FIG. 27 illustrates the same multi-purpose bandages connected In use to support pelvic fractures;

FIG. 28 illustrates the same bandages used to bind the upper legs when immobilising the lower limbs;

FIG. 29 illustrates the bandages being used to similarly bind the lower legs when immobilising the lower limbs;

FIG. 30 illustrates use of the connected multi-purpose bandages to bind the feet together;

FIG. 31 is a front view of an arm bound using interconnected multi-purpose bandages to which a splint is applied;

FIG. 32 is a rear view of the pressure bandage and splint assembly shown in FIG. 31;

FIG. 33 illustrates the use of the multi-function sling strap shown in FIGS. 17 and 18 as a “collar and cuff” type sling;

FIG. 34a is a front view of the multi-function sling assembly in use as an arm sling with the multi-function sling strap in front;

FIG. 34b is a front view of the multi-function sling assembly in use as an arm sling with the sling strap around the back;

FIG. 35a is a front view showing the modular multi-function sling assembly in use as an elevation sling with the sling strap in front;

FIG. 35b is a front view showing the modular multi-function sling assembly in use as an elevation sling with the sling strap around the back;

FIG. 36 illustrates a walking stick constructed from elements of the modular medical support framing system according to the second aspect of the invention as shown in FIGS. 6 to 11;

FIG. 37 illustrates an emergency pole constructed from elements of the modular medical support framing system according to the second aspect of the invention as shown in FIGS. 6 and 7;

FIG. 38 illustrates the use of the multi-purpose bandage to support an ankle sprain or strain;

FIG. 39 illustrates the use of the multi-purpose bandages to support a knee sprain or strain;

FIG. 40 illustrates the use of the multi-purpose bandage to support a wrist sprain or strain;

FIG. 41 illustrates step 1 in producing a support mat using a multi-purpose bandage with pockets with two rods from the support framing system;

FIG. 42 illustrates step 2 in forming the mat being constructed in FIG. 41;

FIG. 43 illustrates the support mat shown in FIGS. 41 and 42 in use with a further multi-purpose bandage to provide wrist support;

FIG. 44a illustrates a first traction splint set up for the left leg constructed from elements of the modular support framing system of the second aspect and the traction strapping system of the sixth aspect of the invention;

FIG. 44b illustrates a second, alternative traction splint set up for the right leg constructed from elements of the modular support framing system of the second, the multi-function strap of the fifth aspect and the traction strapping system of the sixth aspect of the invention;

FIG. 45 illustrates use of the traction splint of FIG. 44a in combination with the traction strapping system of the sixth aspect of the invention and further multi-purpose bandages in accordance with the first aspect of the invention;

FIG. 46 illustrates use of the traction splint of FIG. 44b in combination with the traction strapping system of the sixth aspect of the invention, a multi-function strapping device of the fifth aspect of the invention and further multi-purpose bandages in accordance with the first aspect of the inventions;

FIG. 47a illustrates a splint set up that allows the leg to rest on the platform constructed using multi-function straps in combination with the modular support framing system;

FIG. 47b illustrates the splint set up in FIG. 47a in use;

FIG. 48 shows an alternative splint arrangement using multi-function straps with rods joined by a connector and extending between the strap pockets and in FIGS. 31 and 32 shown with multi-purpose bandages used as an extended bandage;

FIG. 49a illustrates yet another alternative splint using similar components but with a different splint frame;

FIG. 49b illustrates the splint set up of FIG. 49a in use;

FIG. 50a illustrates an example of a splint use to support an ankle injury;

FIG. 50b illustrates the splint set up In FIG. 50a in use;

FIG. 51a illustrates a splint support arrangement similar to that shown in FIG. 50 but configured to support a knee injury;

FIG. 51b illustrates the splint set up in FIG. 51a in use;

FIG. 52a illustrates a complex splint set up for holding the arm in place above the head; and

FIG. 52b illustrates the splint set up in FIG. 52a in use.

### PREFERRED EMBODIMENTS OF THE INVENTIONS

Pressure and/or Support Bandage

The present invention relates to a kit form multi-purpose trauma management system that comprises a number of novel individual elements that are adapted to interact in various ways to provide a vast number of trauma treatment solutions that can each be tailored to a particular intervention.

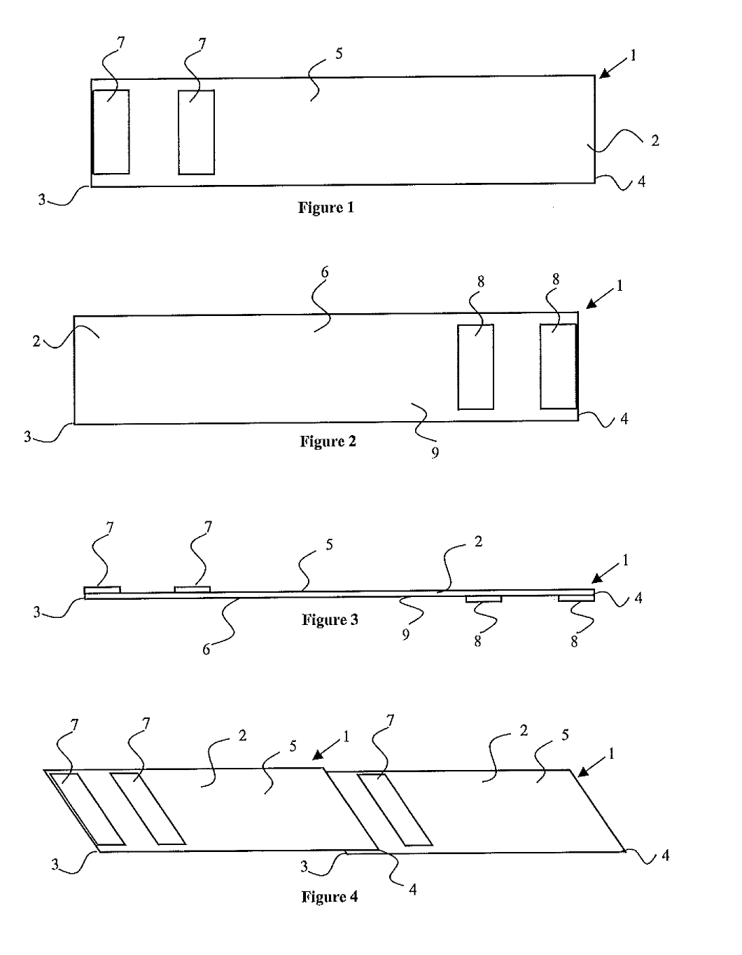

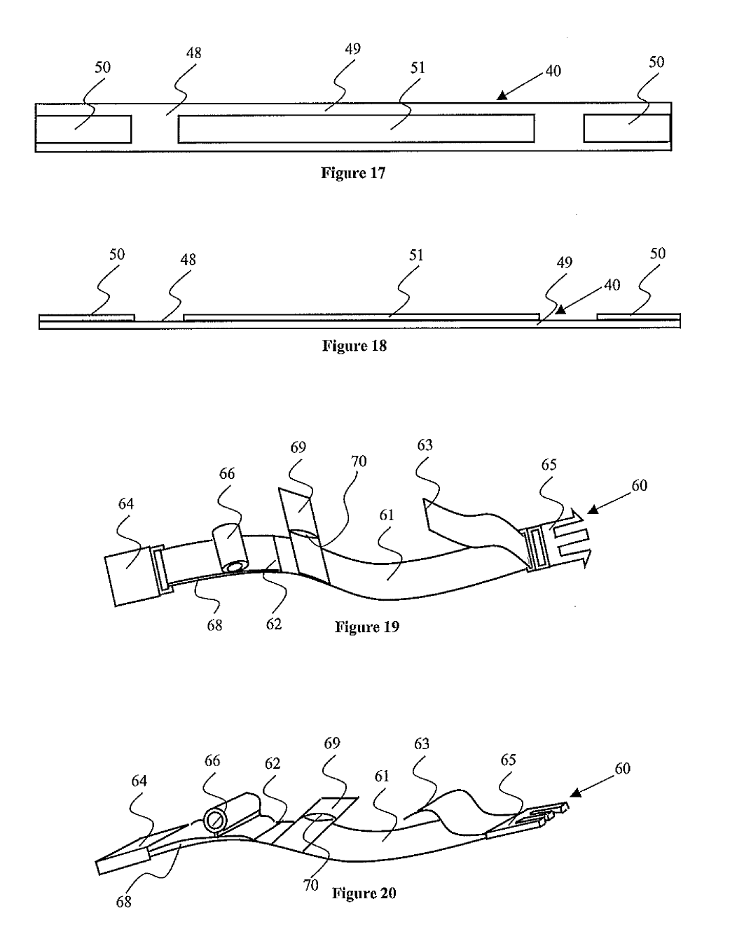

The individual elements will first be described with reference to FIGS. 1 to 21. After that, examples of applications of various combinations of these elements will be described in reference to the treatment of various traumas.

Referring first to FIGS. 1-4 there is shown a first embodiment pressure and/or support bandage according to the first aspect of the invention, hereinafter referred to as a multi-purpose bandage 1.

The multi-purpose bandage comprises an elongate strip of elastically extendable material 2 having a length L extending from a first end 3 to a second end 4 and having a width W perpendicular to the length L. The elastic strip 2 has a first surface 5 on one side and a second surface 6 on the remote side.

Substantially across the width W on the first surface 5 is at least one region of hook material, preferably in the form of two or more strips of hook fastener material 7 at or adjacent, in this instance, the first end 3. Secured to the second surface 6 on the remote side, is at least one, or more preferably two or more, complementary strips of loop fastener material 8 secured at or adjacent the second end 4. The remainder of the second surface 6 comprises predominantly a connectable material 9 that is adapted to engage with the hook fastener material 7.

In a second embodiment shown in FIG. 5, the multi-purpose bandage 1 further includes on its first surface 5 two or more pockets 10 adapted to receive and retain splinting rods and the like as will be described in more detail below. In the embodiment shown the pockets are in longitudinally opposed pairs open at facing edges to receive the splinting rods 11.

Ideally, the width W of the multi-purpose bandage 1 is selected to be wide enough for pressure and/or support applications to large areas including limbs, stomach and chest. In preferred forms the width W is between 100 mm and 250 mm and is more ideally around 150 mm.

Similarly, the length L is preferably selected to suit a combination of limbs circumferences and/or sized for other intended uses such as splinting applications as will be described hereafter. It is currently envisaged that the length L could range from around 400 mm to 900 mm, with the currently preferred length being 500 mm or 600 mm.

The elastically extendable strip 2 can comprise any of a range of suitable materials that provide the requisite combination of resilience, durability, physical support, connectability with loop fastener material on at least one side, and ease of use.

In a preferred form, the material is a knitted waistband or plain knitted support elastic which, by its structure is inherently securable with the hook fastener material strips 7. The currently preferred material is a C930 150 mm wide strong plain knitted support elastic a described by the manufacturer Webbing Industries Australia P/L.

While this preferred material has uniform characteristics across its length and width, the elasticised strip 2 could be of a composite construction fabricated from a combination of different materials or, alternatively, be made of a material that has different properties in different locations and/or on different surfaces. For example, the material may only be hook fastener engagable on second surface 6.

Modular Medical Support Framing System

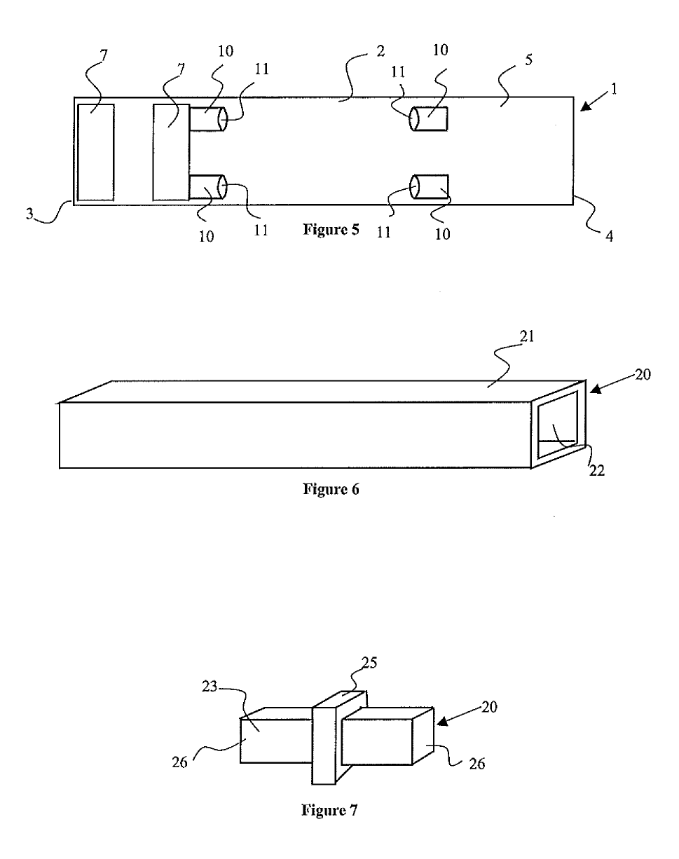

Turning next to FIGS. 6 to 11 there are shown the key elements forming part of a modular medical support framing system 20. In its simplest form, the system comprises a plurality of different lengths of rods 21. In the preferred form illustrated, the rods comprise multiple different length square section tube elements defining corresponding square openings 22 at either end. Also forming part of the system 20 is the plurality of inline connector pieces 23 and corner connector pieces or elbows 24. Each of the connectors 23 and 24 include a centralised stop formation or corner element 25 from which extend two male lugs 26. Ideally each of the lugs 26 are tapered at their distal ends to facilitate initial connection, but sized adjacent the stop formation 25 to provide an interference fit with the openings 22 in the rods 21.

It will be appreciated that alternative rod structures could be used and similarly that different connector structures could be used including connectors configured to connect with the outside surfaces of the rod elements.

The resistance to relative rotation between the interconnected rod elements and the connectors themselves can be achieved purely by selection of the elements inherent shape, as in the case when using square section hollow tubing as in the preferred embodiments, or can be achieved by incorporation of other non-circular connecting faces or locking pins etc.

In a preferred form of the framing system 20, it further includes a plurality of adjustable angle connector pieces 27 such as that shown in FIG. 9 which may incorporate some form of adjustable ratchet type system or suitable alternative as will be known to those skilled in the art.

Also included in preferred forms of the framing system is one or more padded rod assemblies 28 such as that shown in FIGS. 10 and 11. As will be seen, this simply comprises a length of rod 21 that is encased within some form of padding material 29.

Modular Stretcher System

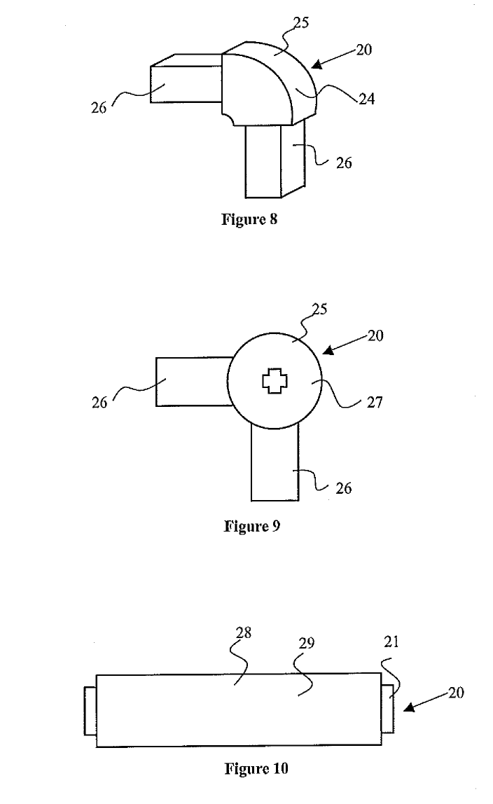

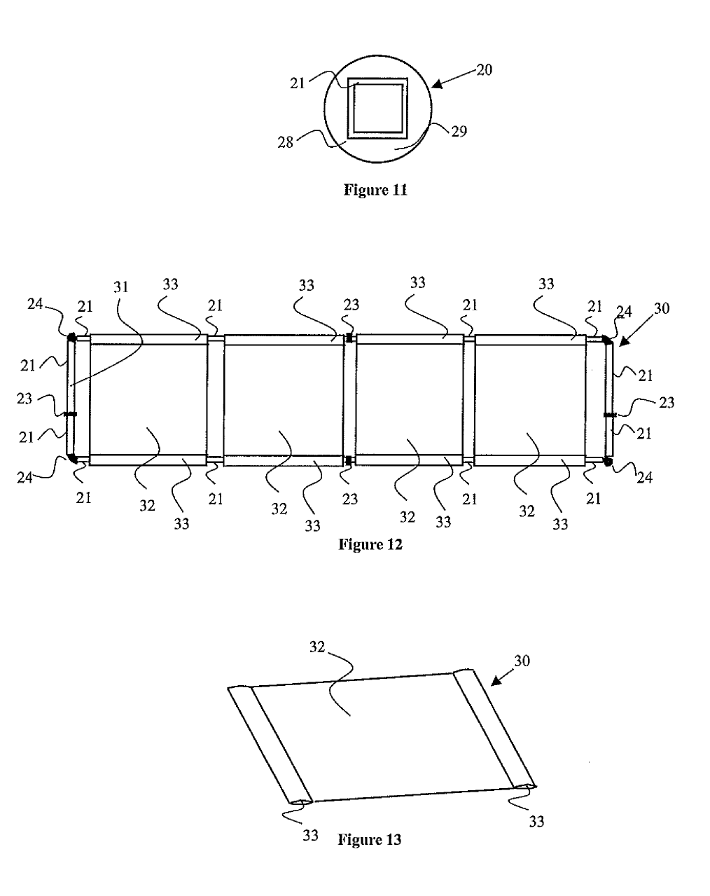

The modular stretcher system 30 as shown in FIGS. 12 and 13 utilises a frame made in the same way as the modular support framing system just described. Essentially, it comprises a plurality of rods or tubes 21 of the kind shown in FIG. 6, interconnected by a plurality of inline and corner connector pieces 23 and 24 as shown in FIGS. 7 and 8. These items are connected to form a generally rectangular frame 31. During assembly, two or more stretcher sling elements 32 are connected. The sling element 32 comprises a sheet of support material having a width corresponding to the width of the rectangular frame 31 and a length less than the maximum length of the longest intended length of the rectangular frame. The sling element 32 as shown in FIG. 13 includes peripherally located longitudinally extending pockets 33 through which the longitudinal rectangular frame elements or rods 21 extend. In this manner a variable length of stretcher can be created by utilising more or less rod elements along the frame length and then selecting the appropriate number of sling elements to suit the creative frame length.

In one preferred embodiment, the stretcher kit includes the following components for an adult-sized stretcher (fewer components being required for a child-sized stretcher):

4×Stretcher slings;

14×Rods @ 280 mm;

2×Rods @ 137.5 mm;

4×Corner connectors; and

12×Inline connectors.

In a preferred embodiment, the stretcher kit is provided as an adjunct to the modular support framing system previously described. In this form, the stretcher kit is designed to utilise at least some of the rod and connector elements of the framing system 20.

Modular Multi-Function Sling Assembly

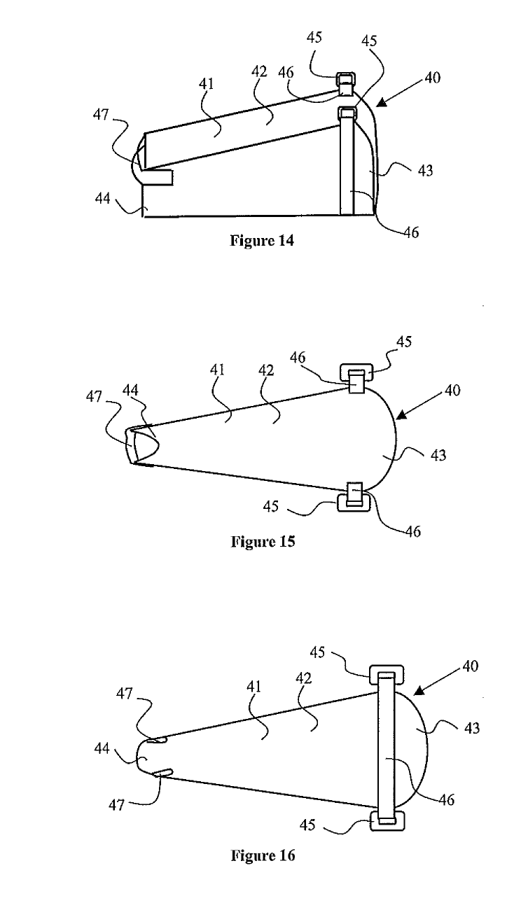

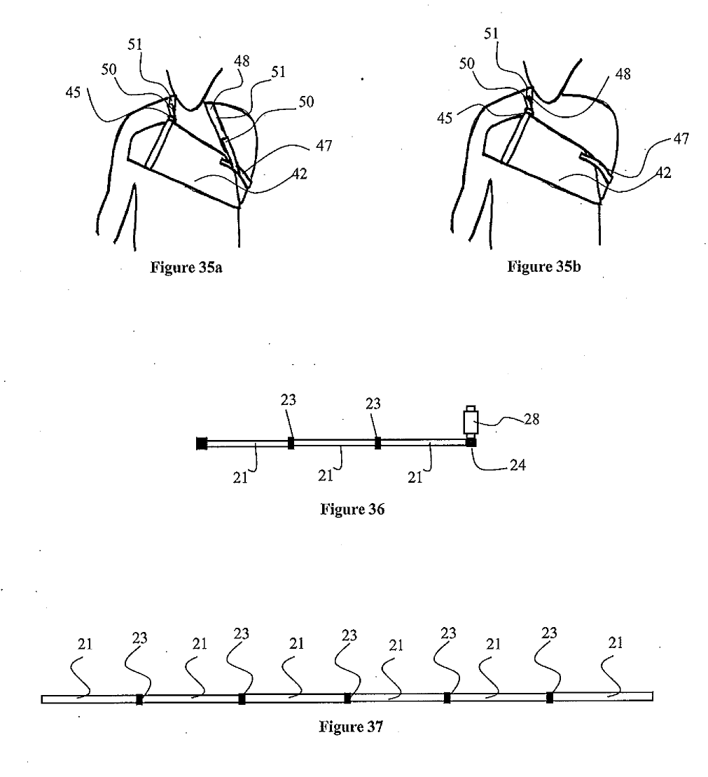

Turning next to FIGS. 14 to 18 there is shown the various elements of a modular multi-function sling assembly 40 in accordance with a fourth aspect of the invention. The sling assembly (as shown fully assembled in FIGS. 34a, 34b, 35a and 35b) comprises a shaped element of ideally lightweight reliable support fabric 41 foldable about a longitudinal axis to form a support pocket 42 as shown in FIG. 14. The pocket defines a first end 43 that is to be located in use or adjacent a wrist, and a second end 44 that in use is to be located at or adjacent an elbow.

The support pocket 42 includes wrist end connection means which in the preferred form illustrated is in the form of pair of loops 45 to which a strap can be secured as described in more detail below. Preferably, the loops are secured to the pocket 42 by means of webbing 46 which extends across the full width of the support fabric 41 as shown thereby also acting to reinforce the support pocket.

In the illustrated embodiment the elbow connection means are in the form of a strip 47 of webbing material or similar which connects the elbow end edges of the support fabric 41. Ideally this is sufficiently long to enable connection of a strap at any convenient point closer to the front or rear of the pocket 42 as needed.

The separate sling strap element 48 is preferably constructed so that it is independently fastenable at each end to itself so that it can also operate in isolation to provide additional functions such as serving as a “collar and cuff” sling as described in more detail hereafter.

In the preferred form illustrated the strap 48 is made of a webbing material 49 or similar and incorporates on one surface relatively short lengths of one of a hook or loop fastening material 50, with discrete or continuous lengths of the other of said hook or loop fastener material 51 disposed in the gap there between as shown. Preferably the outer short lengths 50 are hook fastener material and the lengths in the gap between are loop fastener material. In a variation of this embodiment the webbing material itself may have a surface which is connectable with hook fastener material.

In an alternative embodiment, the fasteners on the strap can be buckle arrangements such as double ring connectors or similar disposed and configured to allow length variation of the strap at one or both ends.

It will be appreciated that by having an independently useable strap and a specially configured support pocket that is readily rolled up for transport, the multi-function sling assembly can perform more functions, enable the strap to be secured either over the arm to provide some restriction of movement, or behind the shoulder to reduce pressure on the injury if required, and take up less space than prior art dedicated structured sling assemblies, particularly those heavy or rigid formed pockets or with integral straps.

Multi-Function Strap that can Operate as a Tourniquet

Referring next to FIGS. 19 and 20 there is shown an example of a multi-function strapping device 60 that can also act as a tourniquet in accordance with the fifth aspect of the invention. The strapping device comprises a length of substantially inextensible material, such as a webbing material 61, defining in use a first end 62 and a second end 63. Secured to the first end 62 is a female clip socket 64. A slidably repositionable male clip element 65 is provided at a location between the first and second ends. Adjacent, but spaced from the female socket 64, is a rod receiving loop 66.

Preferably, the female clip socket 64 is secured to the webbing 61 by passing an end 62 of the webbing through the socket connector loop and connecting that end back on an upper surface of the webbing a short distance from the female socket. In this way a webbing loop 68 is formed such that during twisting tightening of the tourniquet using a rod in the rod receiving loop 66, the webbing in the webbing loop 68 can move through the connector loop in the female clip socket 64 and so reduce the risk of pinching.

In the preferred form illustrated the strapping device 60 further includes one or more pocket formations 69 having an opening 70 to receive a splinting rod such as that shown in FIG. 6.

Traction Strapping System

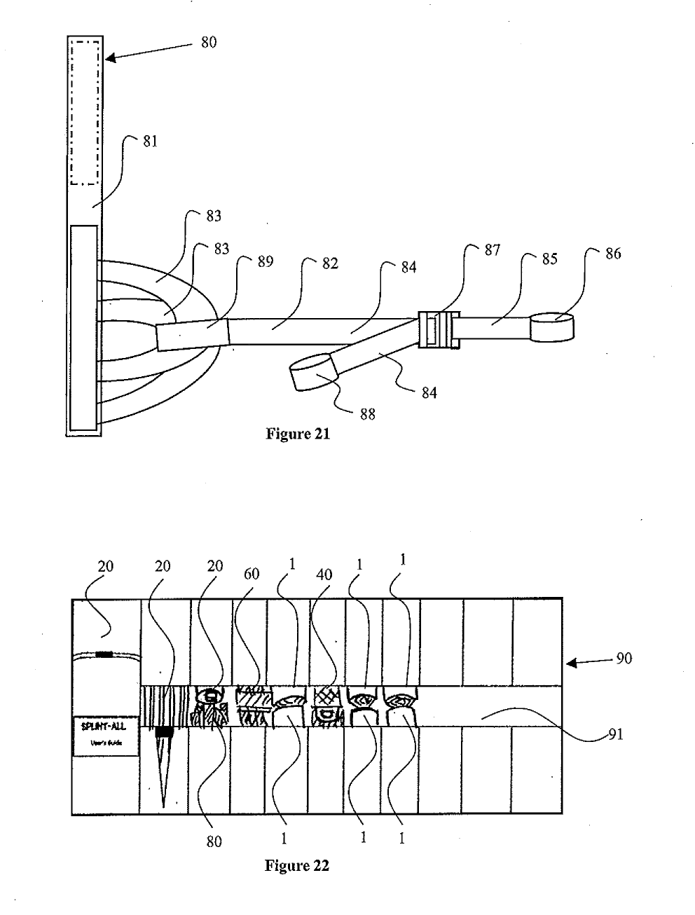

FIG. 21 illustrates a first embodiment traction strapping system 80 in accordance with the sixth aspect of the invention. The strapping system includes a releasably securable limb cuff arrangement shown generally at 81 that is adapted for securing around a limb joint, and more preferably a distal limb joint, such as an ankle or wrist. Connected with this cuff arrangement 81, is at least one tensionable length adjustable traction strap assembly 82. In the illustrated embodiment, the strap assembly 82 includes a plurality of connector straps 83 which form a loop or series of loops, each end being secured to the cuff 81 and passing through a fixed loop 89 at the proximal end of the length adjustable strap 84 to connect the cuff arrangement 81 to the adjustable strap 84 via the connector strap or straps 83. In the currently preferred form the main strap comprises a fixed length distal portion 85 that ideally terminates in a connector formation in this case in the form of a loop 86 which acts as a means for connecting the strap assembly to a fixed structure such as a specially configured splint or frame.

The remaining portion of the main strap 84 intermediate the proximal portion 85 and the connector straps 83 secured to end portion 85 via passage through a sliding lock 87 rigidly connected to the proximal end of strap portion 85. As can be seen, the adjustable strap 84 folds back on itself so as to extend back toward the cuff arrangement 81, thereby enabling application of tension by the victim if required. The free end of strap 84 preferably terminates in another connector formation such as the illustrated loop 88 so that extension straps and the like can easily be connected to help application of the tensional force if needed.

Modular Trauma Management System

Referring next to FIG. 22 there is shown one preferred form of the modular trauma management system 90 of the seventh aspect of the invention. The kit in its most basic form includes various multi-purpose bandages in accordance with the first aspect of the invention as described with reference to FIGS. 1-5 in combination with items of the modular medical framing support/splinting system of the second aspect of the invention discussed with reference to FIGS. 6-11.

However, in the preferred form the kit includes a combination of all of the elements of at least FIGS. 1-11 and 14-21, optionally extended to facilitate construction of a stretcher as shown in FIGS. 12 and 13.

Preferably the kit is provided in a pocketed carrier formation 91 such as that shown in FIG. 22 which can be rolled into a compact bundle for easy transportation. As will be seen additional pockets may be provided for inclusion of additional first aid items such as gauze pads, ointments, scissors, saline and the like as known and/or needed.

In the currently preferred form of the system it includes the following components:

5×multi-purpose bandages;

1×multi-purpose bandage with pockets;

6×rods @ 280 mm;

3×rods @ 137.5 mm;

2×rods @ 66.25 mm;

1×padded rod @ 150 mm length;

6×inline connectors;

6×corner connectors;

1×traction strapping system;

3×multi-function tourniquet straps;

1×multi-purpose sling strap; and

1×sling support pocket.

This kit of components in the roll weigh only 1.5 kgs and rolls up into a cylindrical package of a size 30 cm high and 15 cm diameter or a volume of approximately 5.3 litres. This makes it ideal for carrying to remote locations where vehicular support cannot be used, and a package of existing currently available equipment capable of performing equivalent functions would simply be far too heavy and cumbersome to be realistically considered.

The kit may also comprise several angle adjustable connectors to provide further versatility where complex angled splints need to be made.

Further, if it is desired to extend the kit to have the capability of constructing an adult-sized stretcher in accordance with the third aspect of the invention, only the following additional lightweight components need be included:

4×Stretcher Sling Elements;

8×Rods @ 280 mm length; and

6×inline connectors.

It will be appreciated that these quantities can be varied, additional components can be added, or certain components omitted, each according to the specific requirements and/or proposed applications.

Use of the Various Components of the Modular Trauma Management System

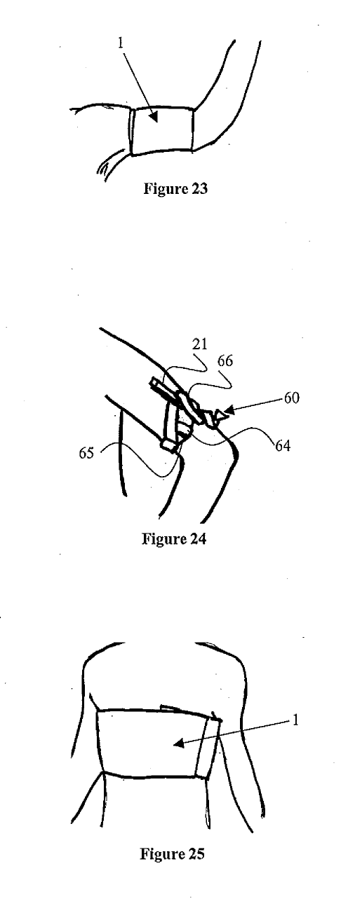

Referring next to FIGS. 23-52b there is shown a selection of trauma situations to which the various elements of the modular trauma management system can be applied and the method of application. It will of course be appreciated that numerous additional uses are possible beyond those illustrated and that the following is intended purely to give some specific examples to demonstrate the versatility of the invention in use and indicate how various types of trauma may be treated.

Managing Bleeding

The multi-purpose bandages 1 can be used to apply pressure to wounds in many bleeding situations. To do this appropriate padding is placed against the wound site. The padding is then held in place by applying and securing an appropriate width or length multi-purpose elasticised bandage to the affected region.

FIG. 23 shows a single multi-purpose bandage I secured over an upper arm region. In this case only a single strap is initially required. The multi-purpose bandage 1 is positioned with the hook fastener strips facing inwards at the external and uppermost aspect of the multi-purpose bandage 1. Once wrapped around the arm with the requisite tension, the hook fasteners can be secured to either the hook engagement remote side of the elasticised material, or the strip or strips of loop fastener material, depending on the size of the arm. If additional pressure or securement is required, the multi-purpose bandage 1 can be refastened more tightly without releasing the existing tension or a further bandage can optionally be applied on top.

Similarly, if it is necessary to apply a tourniquet at or adjacent the affected region, the multi-function tourniquet strap 60 can be applied proximally to, or if needs be over, the multi-purpose bandage. An example of this is shown in FIG. 24 without the bandage for clarity. To do this the strap 60 is wound around the arm, secured by inserting the male clip 65 into the female clip socket 64 and tightened initially by pulling the free end of the strap. A rod, such as one of the rods 21 is then inserted in the rod loop 66 and twisted to further tighten the strap. The twisted rod is then secured using the free end of the strap to retain the tension. A further strap could also be used.

Cracked Rib

A cracked rib is best managed with gentle support which can be conveniently provided with a belt formed from interconnected multi-purpose bandages 1 as shown in FIG. 25. The bandages are first interconnected as shown in FIG. 4 and then wound around the chest to provide a supporting elastic splint and secured as previously described.

Abdominal Injury

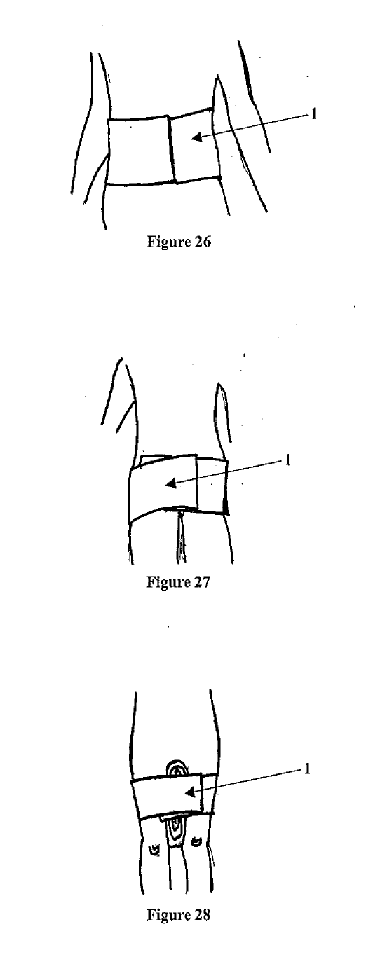

Relief can often be provided for abdominal injuries using gentle support of the kind described above in relation to the cracked rib and as illustrated in FIG. 26.

Pelvic Fractures

In some instances pelvic fractures can result in the patient feeling as though the pelvis is “coming apart”, in which case an elasticised support provided using interconnected multi-purpose bandages as described above may again provide relief as per the example shown in FIG. 27.

Leg Injuries

Many injuries to the lower leg limbs can be managed by using one leg as a splint against the other. To do this padding is first placed between the legs prior to splinting to maintain comfort. Again the support is provided using joined multi-purpose bandages 1 as described above.

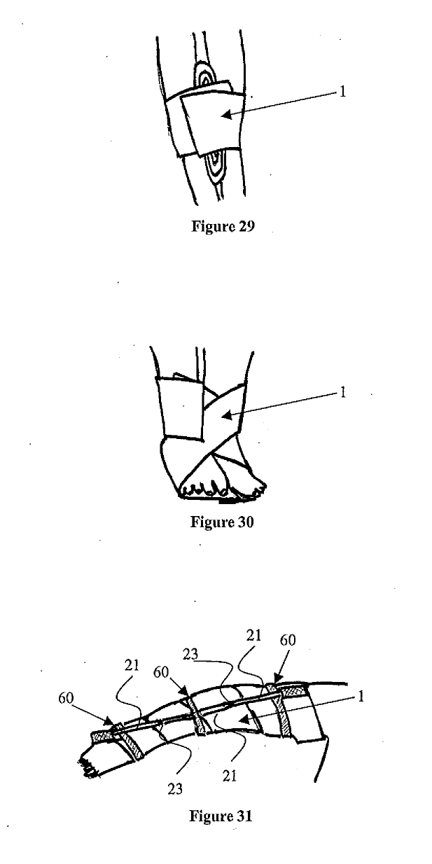

It is recommended that if using this technique the upper legs (FIG. 28), lower legs (FIG. 29) and feet (FIG. 30) are all bound to provide maximum splinting effect.

Envenomation

Most bites, particularly to limbs, are best managed with a pressure immobilisation bandage. To make the bandage, you once again simply apply single, or multiple joined, multi-purpose bandages around the affected limb, applying firm pressure all the way from the limb tip to the torso.

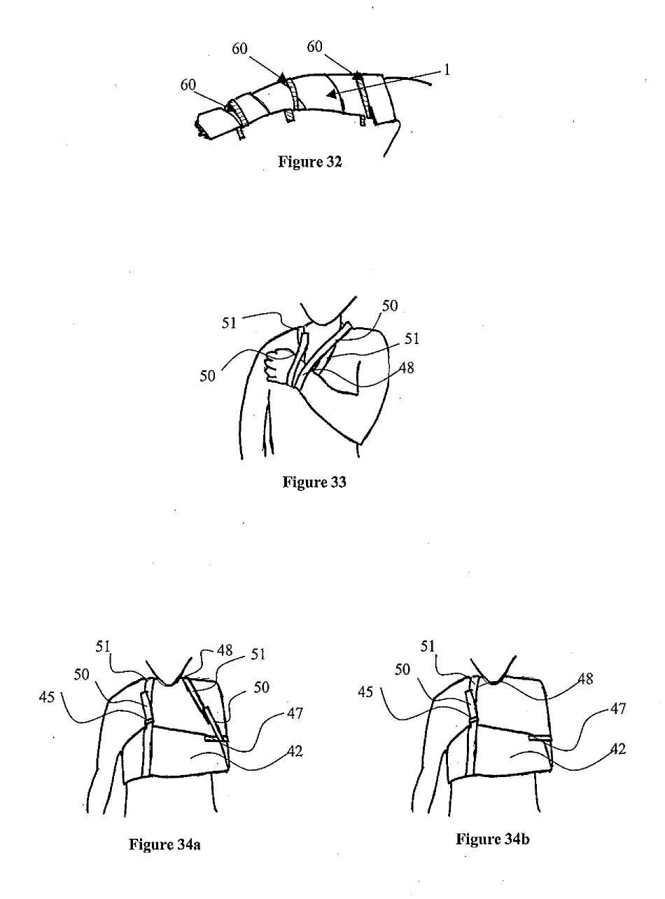

To further immobilise the limb it can be splinted either by splinting to the body if an arm, or to the other leg if the injury is on the leg. Alternatively, a simple external splint could be employed. An example is shown in FIGS. 31 and 32. As can be seen, a pressure immobilisation bandage has been applied using several joined multi-purpose bandages 1. A simple splint is then constructed by joining appropriate rod lengths 21 with inline connectors 23. The splint can then be secured to the arm using two of the pocketed multi-function tourniquet straps 60 which serve to retain the ends of the assembled splint rod. Further straps may be provided between the ends as needed.

Arm and Shoulder Injuries

Many arm and shoulder injuries can be managed, or at least assisted, by using a sling. There are three types of sling that can all easily be made and even self applied using the components of the multi-function sling assembly,

Collar and Cuff Sling

This form of sling can be made very simply using solely the sling strap element 48 as shown in FIG. 33. This strap is first draped around the injured person's shoulders with the ends resting on the chest, and the hook fastener material 50 near the ends facing outward. The hand is then raised to the desired height up near the shoulder. Each end of the strap is then folded back over the wrist such that the hook material strips 50 can each fasten to the loop material or hook material engageable material 51 on that same side. This is fast, simple and easy to adjust.

Arm Sling and Elevation Sling

By using the strap element 48 with the sling pocket 42 you can make either an arm or elevation sling. First place the sling pocket in front of the patient so that the elbow section of the pocket is at the patient's elbow and secure one and of the strap 48 around the elbow connection strip 47. Place the arm on the injured side in the sling pocket and the loose end of the strap 48 is then placed around the neck and secured to the wrist end connection loops by passing through both loops 45 and again securing the free end back to the strap using the hook and loop fasteners as described above. The height of the wrist can be adjusted to provide either an arm sling (as in FIGS. 34a and 34b) and or an elevation sling (as shown in FIGS. 35a and 35b). Similarly the location of the strap from the neck to the elbow can be adjusted to be either in front as shown in FIGS. 34a and 35a, or around the back as shown in FIGS. 34b and 35b, whichever is most comfortable for the nature of the injury sustained,

Injuries Requiring a Walking Stick or Stretcher Pole

When needed a comfortable fully functional walking stick can be made using various elements of the modular medical support framing system by simply connecting various rods 21 with inline connectors 23 to get the desired length and then finishing with an elbow connector 24 and padded rod 28 as shown in FIG. 36.

Also, by simply joining connector rods 21 with inline connectors 23 you can fabricate a suitably sized simple pole for one side of a blanket stretcher, or other emergencies as shown in FIG. 37.

Sprains and Strains

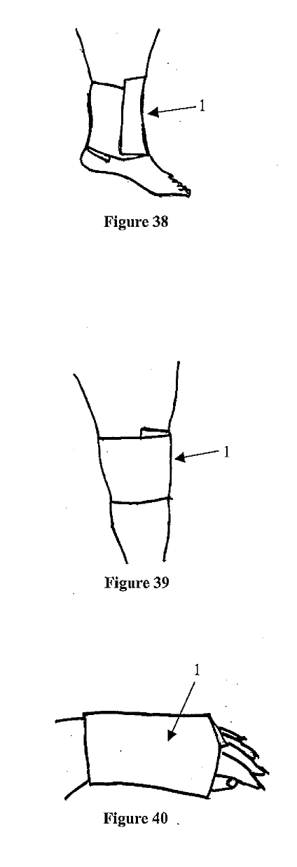

Sprains and strains can often feel much improved with some simple support, and once again this can be provided by using the multi-purpose elasticised bandages 1 around and, optionally, above and below the injury site as shown in FIGS. 38, 39 and 40.

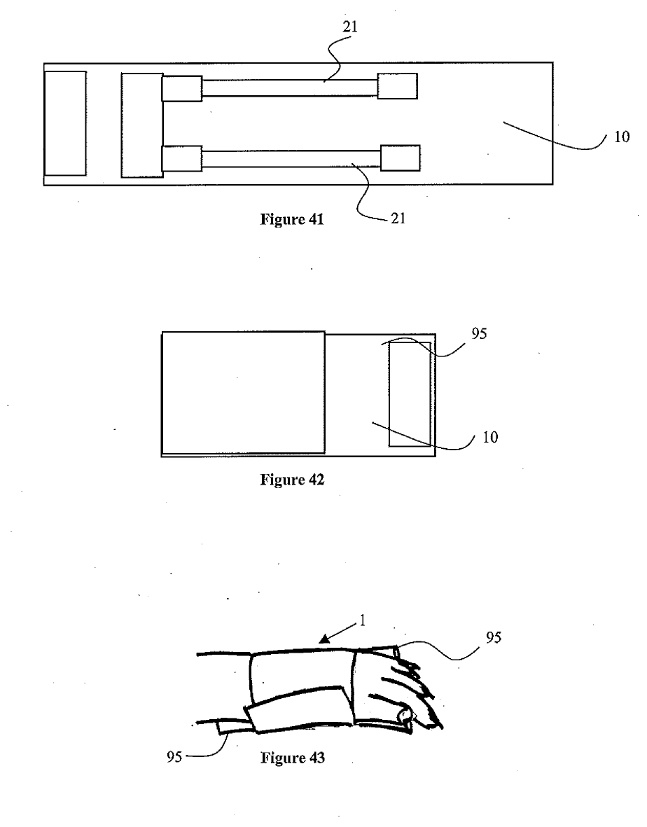

Splinting for a Lower Arm Injury

Where additional support is required for a lower arm injury than cannot be provided solely with a sling, a support mat 95 can be produced using the multi-purpose bandage 1 that includes the integral pockets 10 which can hold and retain two rods 21 as shown in FIG. 41. Once the rods are assembled, the ends of the bandage either side of the rod ends are folded over and secured against each other to form the support mat as shown in FIG. 42. This support mat 95 can then be placed under the injury site and gently wrapped with a further multi-purpose bandage 1 as shown in FIG. 43.

This procedure can be varied for an upper arm injury where it is likely to provide additional comfort.

Splinting for an Upper Leg Fracture

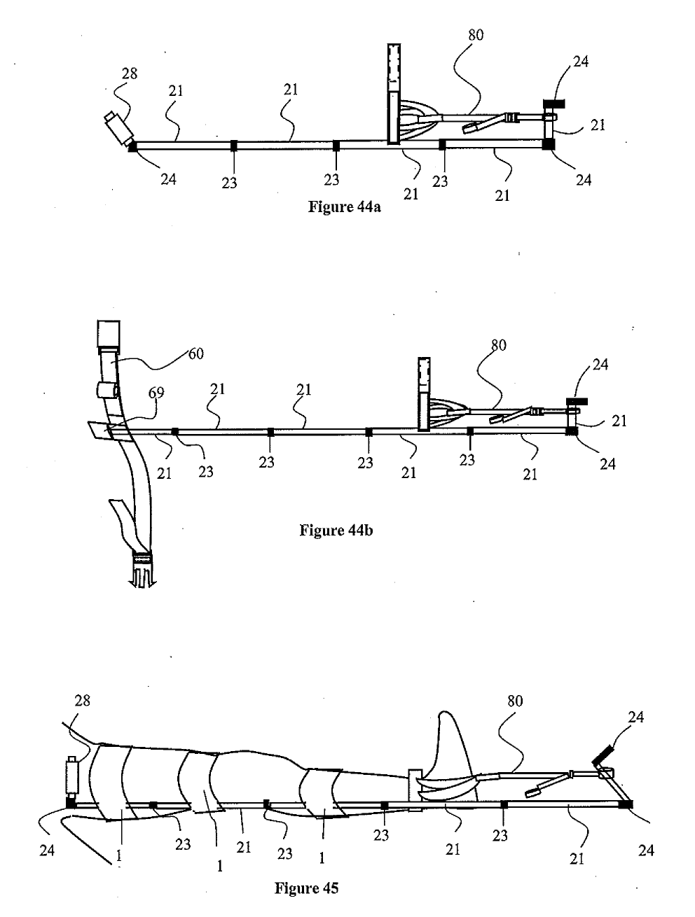

Fractures of the mid and lower shaft femur are often managed with traction. To this end a traction splint as shown in FIGS. 44a and 44b can readily be devised using the traction strapping system 80, several multi-purpose bandages 1, and various rods 21, connectors 23 and 24 of the framing system 20.

You can start with the padded rod 28 and insert corner connector 24. Long rods 21 are then joined using connectors 23 to build a continuous rod that is approximately 300 mm longer than the patient's straightened good leg from hip to foot. A corner connector 24 is then added and a short rod 21 so that the short rod will sit behind the heel of the injured leg when the padded rod is placed in the groin region.

Once the splint is fabricated, the cuff element 81 of the traction strapping system is carefully placed around the ankle of the injured leg and fastened using the preferred hook and loop fasteners provided thereon as shown in FIG. 45. The connector straps 83 need to be carefully run evenly down each side of the foot and the main strap 84 adjusted to be as long as possible ideally leaving the connector formation in the form of the pulling loop 88 up against the sliding lock 87.

The padded rod 28 is carefully and gently placed as high as possible on the injured side into the groin of the injured person.

Moving next to the foot end of the assembly, the end connector loop 86 is then slid over the short rod behind the foot and a corner connector 24 inserted at the free end so that the loop cannot slip off. Multi-purpose elasticised bandages 1 are then placed where the leg will be ready to be secured to the injured leg once traction has been applied.

The splint is now set up and the next step is to straighten and traction the fractured leg, initially by hand. Once initial traction has been applied and the leg is straightened further traction can be applied and maintained by tightening the traction strap 84 which can be achieved by pulling on the free end 88, thus drawing the traction strap though the sliding lock 87. Once the desired degree of traction is achieved the remaining bandages 1 are secured around the leg and splint to stop any movement.

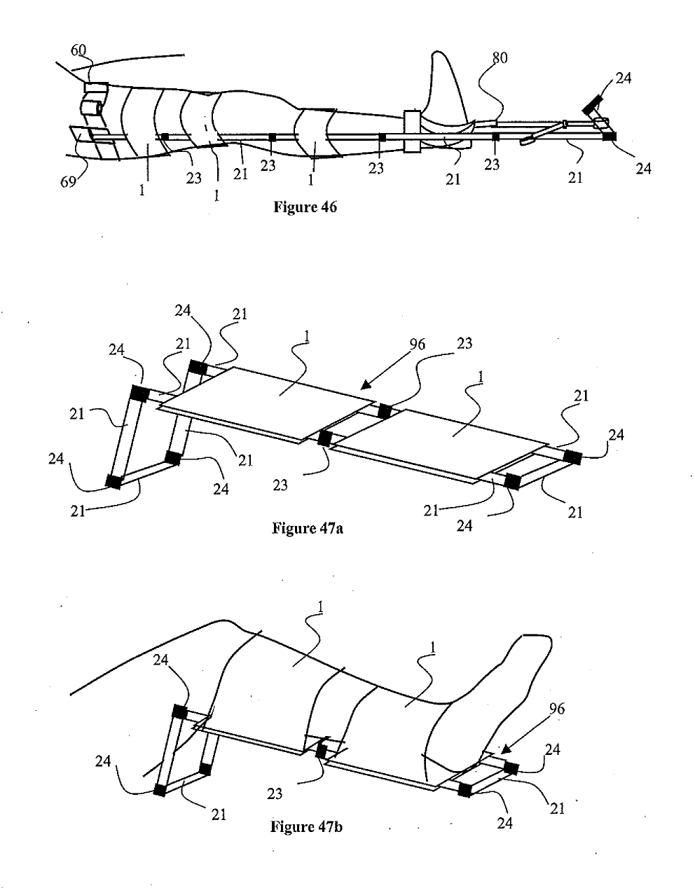

An alternative thigh connection is shown in FIG. 46. In this version the pocket 69 from one of the multi-function tourniquet straps with pockets 60 is used at the thigh end of the splint rod to secure the splint to the lateral aspect of the thigh before traction is applied in the same manner as above.

Splinting for a Lower Leg Injury

Using the basic multi-purpose bandages 1 and elements of the modular medical framing support system 20, it is possible to produce a splint set up that provides a platform 96 as shown in FIG. 47a which can be used to support a lower leg from under the knee as shown in FIG. 47b. The construction is self-explanatory and again additional bandages 1 can then be used to strap the leg to the leg platform 96. This platform can be constructed with an extended angled portion adjacent the knee and behind the thigh if it is desirable to provide better support for the knee.

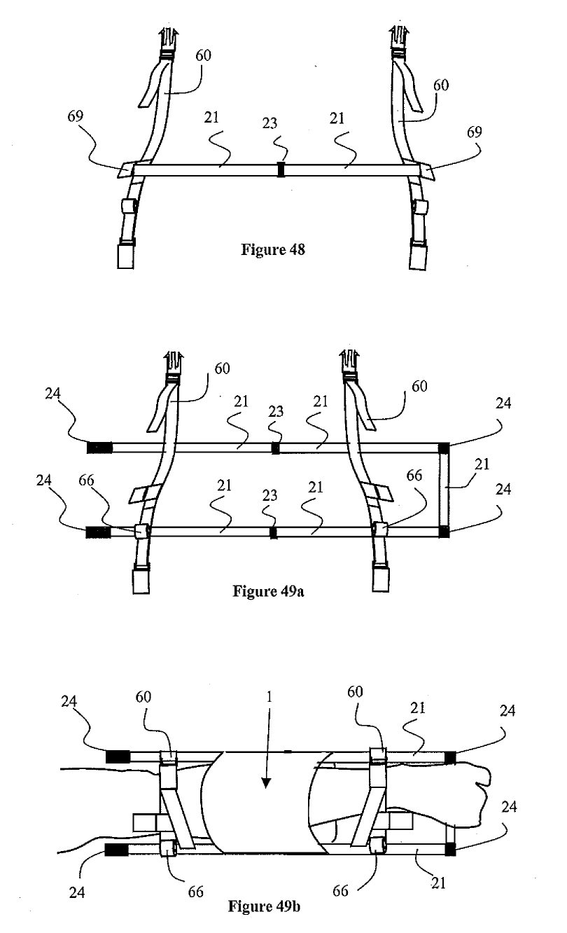

FIG. 48 shows an alternative splint arrangement that would have numerous uses that simply comprises a rod or connected rods 21, held at its ends in the pockets 69 provided on two multi-function tourniquet straps 60.

Yet another variation is shown in FIGS. 49a and 49b, where instead of the rods being held in the closed pockets 69, they instead are retained by passing through the loop 66 on each strap 60 that also doubles as the rod receiving loop for when the strap is to be used as a tourniquet.

Splinting for an Ankle or Knee Injury

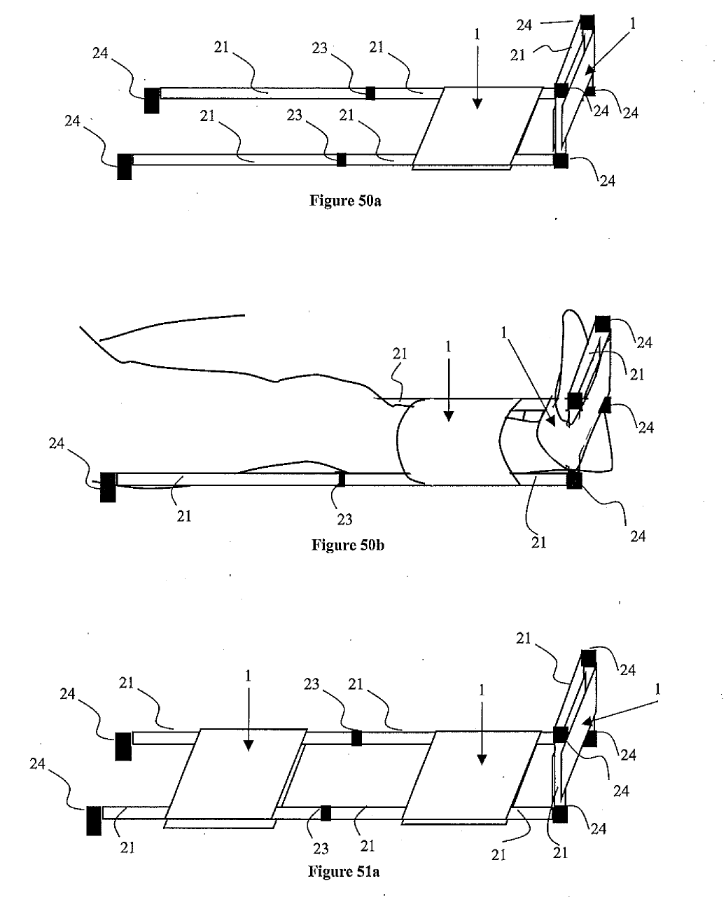

FIGS. 50a and 50b illustrate just one example of a splint that could be used to support an ankle injury. The structure and method of construction can be seen in the drawings and is again based on the same basic components of the multi-purpose bandages 1 in combination with elements of the modular medical support frame system 20. An extended variation that can be used to splint a knee injury is shown in FIGS. 51a and 51b.

Splinting for a Shoulder Injury

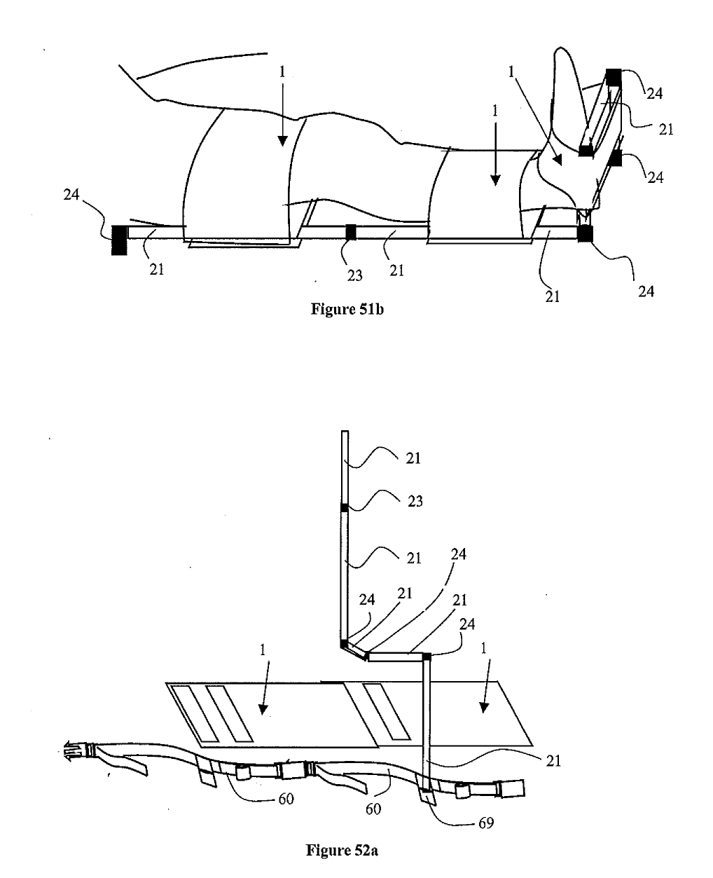

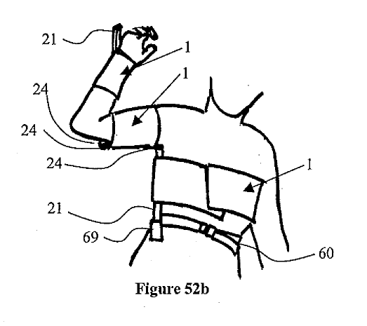

FIGS. 52a and 52b illustrate just one of many complex splint arrangements that are readily possible with the systems of the invention. In this particular instance the splint is designed for a shoulder injury of the kind where it is desirable to stabilise the patient's arm in place above the head.

In such an instance the various length rods and connectors are used to tailor-fit a shape to suit the injured person's presentation. In this particular example the presentation requires the splint to be secured to the body and arm. The splint is secured to the body by using both the pocket 69 in a multi-function tourniquet strap 60 secured around the chest to stop the splint from slipping and multi-purpose bandages 1 wrapped around the rods 21 and chest to hold the splint in place. More bandages 1 can then be used to secure the upper and lower arm to the constructed splint.

Other Uses

It will be appreciated by those skilled in the art that the examples provided are clearly not exhaustive and that the very nature of the modular system and elements of that system provide incredible flexibility which allows a user to tailor an ideal solution for most eventualities.

Advantages

The numerous advantages of the full trauma management system and each of the individual elements are discussed in part and/or are evident from the text above. However, it will be appreciated that not only does the modular trauma management system of this aspect of the invention, by virtue of the adaptability and interaction of the components, enable a compact, lightweight and highly adaptable kit that is easily transported, the nature of each of the preferred elements is such that the entire system is unaffected by environmental conditions such as water, altitude and heat and so, unlike many existing systems, can be used almost anywhere. For example, unlike most conventional bandages which are often unusable when wet, the multi-purpose elasticised bandages of the first aspect of the invention are unaffected by water. Similarly, while air splints and the like can be affected by altitude, the modular rigid rod or tube-based support framing system is not. Similarly, by having an independently useable strap and a specially configured support pocket, the multi-function sling assembly can perform more functions, enable the strap to be secured either over the arm to provide some restriction of movement, or behind the shoulder to reduce pressure on the injury if required, and take up less space than prior art dedicated structured sling assemblies with integral straps. The same applies to the multi-function strap that can act not only as a tailored tourniquet, but also be used to retain splints and bandages and indeed any other suitable strapping functions. As for the traction strapping system, this, like the sling, is designed for easy use by the injured person, which makes it particularly appealing to individuals or small groups venturing to isolated regions who may need to help themselves before external assistance can be found. The first aspect, second aspect, forth aspect, fifth aspect and sixth aspect of the invention improve a person's ability to self-apply the required intervention for traumatic situations as discussed. The third aspect and seventh aspect of the invention, by providing robust modular and multi use components in a lightweight and compact package, improve the possibility of the necessary equipment either being on hand or made available (eg: via helicopter drop) in situations where limited space and/or weight restrictions would otherwise limit capacity to have the equipment available.

Variations

It will also be appreciated that for all aspects of the various inventions, variations beyond those described and alluded to will be possible within each aspect without departing from the spirit and scope of each invention.

1. A pressure and/or support bandage comprising:
an elongate strip of elastically extendible material having a length L extending from a first end to a second end and a width W, with a first surface on one side and a second surface on the remote side;
at least one region of hook fastener material or equivalent extending substantially across the width W at least partially on the first surface at or adjacent one of said first or second ends; and
a complementary at least one region of loop fastener material or a connectable material adapted to engage with said hook fastener material extending substantially across the width W at or adjacent the other of said first or second ends on the second surface on the remote side; the majority of the remainder of said second surface generally being of a connectable material adapted to engage with said hook fastener material;
whereby in use the bandage can be wound around an article and secured by connection of one or more of the hook fastener regions or equivalent on the first surface, with one or more of the complementary loop fastener regions on the remote surface and/or said connectable material, or alternatively, extended in length by overlapping end to end connection of a like bandage via engagement of the at least one hook fastener region on one bandage with the corresponding complementary loop fastener or connectable material region on the remote surface of the other bandage.

2. A bandage according to claim 1, wherein the hook and/or loop fastener regions are in the form of strips of hook or loop fastener material respectively.

3. A bandage according to claim 2, including at least two spaced strips of hook fastener material on the first surface at one end, and a corresponding at least two similarly spaced strips of loop material on the second surface at the other end.

4. A bandage according to claim 1, further including one or more pockets adapted to receive and retain an end of one or more rigid elongate supports.

5. A bandage according to any one of the preceding claims, wherein the elasticised material is a knitted waistband or plain knitted support elastic.

6. A modular medical support framing system adapted for creating a multiple range of splints, support frames and crutch type devices, the framing system comprising;
a plurality of different lengths of rods or tubing adapted at each end shaped to receive an inline connector piece or corner connector; and
a plurality of inline and corner connector connectors;
wherein the inline and corner connectors and/or rod ends are configured to prevent relative rotation of interconnected rods or tubing after connection.

7. A framing system according to claim 6, further including at least one adjustable angle connector.

8. A framing system according to claim 7, wherein the tubes are made from square section aluminium having an external size of approximately 16 mm and a wall thickness of approximately 1.5 mm.

9. A framing system according to claim 6, wherein the connectors include a centralised corner element or connector stop formation from which extend two male lugs designed to provide an interference fit with the square hollow ends of the square section tube elements.

10. A framing system according to claim 6, wherein three lengths of rod/tubing are provided, the first being between 250-300 mm, the second being between 120-150 mm and the third being between 50-75 mm.

11. A framing system according to claim 6, further including at least one padded rod having a length of approximately 150 mm.

12. A modular trauma management system including:
multiple fresher and/or support bandages in accordance with any one of claims 1 to 5;
in combination with
a modular medical support framing system in accordance with any one of claims 6 to 11.

13. A trauma management system according to claim 12, further including a multi-function strapping device that can act as a tourniquet, the strap comprising:
a length of substantially inextensible material having a first end and a second end remote the first end;
a female clip socket secured to a first end;
a slidably repositionable male clip element provided at a location between the first and second end; and
a rod receiving loop formation between said female and male socket and clip;
whereby the length of substantially inextensible material is selected, such that in use as a tourniquet after connection and tightening of the strap via operation of the clip elements, a suitable rod is inserted in the rod receiving loop and rotated to further tension the strap, the excess strap length can be used to secure the rod to retain the tension.

14. A trauma management system according to claim 12 or claim 13, further including a modular multi-function sling assembly, the assembly comprising:
a support pocket having a first end to be located in use at or adjacent a wrist and a second end to be located in use at or adjacent an elbow;
the pocket including wrist end connection means and elbow end connection means; and
a separate strap element, the strap being adapted at each end for releasable connection with each of the wrist end and elbow end connection means.

15. A trauma management system according to claim 12, further including a lightweight modular dismantlable length variable stretcher system comprising;
a plurality of rod or tubing elements adapted at each end to receive an inline connector piece or corner connector;
a plurality of inline and corner connectors; wherein the inline and corner connectors and/or rod ends are configured to prevent relative rotation of interconnected rods or tubing after connection;
the rod or tubing elements being interconnectable via the connectors to form a generally rectangular stretcher frame; and
two or more stretcher sling elements each having a width corresponding to the width of said rectangular frame and a length less than the maximum length of the longest intended length of the rectangular frame and including peripherally located longitudinally extending pockets through which the longitudinal rectangular frame elements can extend;
whereby two or more lengths of stretcher can be created by utilising more or less rod elements along the frame length and selecting the appropriate number of sling elements to suit the created frame length.

16. A trauma management system according to claim 12, further including a traction strapping system comprising:
a releasably securable limb cuff arrangement for securing around a distal limb joint; and
at least one tensionable length adjustable traction strap assembly extendable away from said distal limb joint and terminating in a first connector formation for securement to a fixed frame element,
whereby the traction strap assembly is length adjustable by means of the strap passing through a sliding lock such that the free end of the strap then extends back towards the cuff element and terminates in a loop formation by which traction can be directly or indirectly applied.
